(19) United States
(12) Patent Application Publication
(10) Pub. No.: US 2020/0224239 A1
(43) Pub. Date: Jul. 16, 2020

(54) FUNCTIONAL ANALYSIS OF CANCER CELLS

(71) Applicants: Massachusetts Institute of Technology, Cambridge, MA (US); Dana-Farber Cancer Institute, Inc., Boston, MA (US)

(72) Inventors: Keith Ligon, Brookline, MA (US); Scott R. Manalis, Portland, OR (US); Mark M. Stevens, Cambridge, MA (US); Robert J. Kimmerling, Cambridge, MA (US)

(73) Assignees: Massachusetts Institute of Technology, Cambridge, MA (US); Dana-Farber Cancer Institute, Inc., Boston, MA (US)

(21) Appl. No.: 16/739,866

(22) Filed: Jan. 10, 2020

Related U.S. Application Data
(60) Provisional application No. 62/790,799, filed on Jan. 10, 2019.

(51) Int. Cl. C12Q 1/02; C12Q 1/6886; G01N 1/28

(52) U.S. Cl. CPC C12Q 1/02; G01N 1/28; C12Q 1/6886

(57) ABSTRACT

The invention provides devices and methods for measuring how living cells function. The measurements can be made from tissue biopsy samples to measure functional properties of living cells from a solid tumor. After measuring a functional property of a cell, the cell remains alive and is available for other subsequent analyses. In certain aspects, the invention provides a method for measuring a cancer marker. The method includes obtaining a tissue sample comprising living cells, disaggregating the tissue sample and loading individual live cells into an input channel of a measurement instrument, and flowing the live cells through the measurement instrument to measure a functional property of the live cells.

### RELATED APPLICATION

This application claims priority under 35 U.S.C. § 119(e) to U.S. Provisional Application Ser. No. 62/790,799, filed Jan. 10, 2019, the entire contents of which are incorporated by reference herein.

### TECHNICAL FIELD

The disclosure relates to methods for evaluating disease.

### BACKGROUND

Cancer is a global health issue that causes millions of deaths worldwide every year. Chemotherapy and/or radiation are the traditional treatment modalities for cancer. Most chemotherapeutics target all dividing cells with the hope that the cancer cells will be most susceptible to treatment. While there are some efforts made to tailor chemotherapy to specific tumor types, therapeutics choices are limited. Consequently, treatments may benefit some patients but others receive little or no benefit, and may experience adverse reactions Accordingly, while there is some effort to tailor treatment, there is limited ability to effectively predict how an individual patient will respond to a particular treatment, which may lead to extended periods of time in which a patient endures a treatment that simply isn't working as intended. There are known cancer biomarkers that are associated with disease type and outcome and can drive therapeutic choice. However, traditional methods for measuring those biomarkers are not done on live cells and, therefore, do not take into account the dynamic nature of the cells over time.

### SUMMARY

The invention provides devices and methods for in vitro functional measurement on living cells. Measurements made on living cells reveal not only the presence of cancer cells, but also elucidate the functional aspects of those cells over time, including their response to prospective therapeutic intervention. Measurements made on living cells according to the invention provide a real-time, dynamic view of the cells, enabling more accurate diagnosis and more effective therapeutic selection.

Measurements according to the invention typically are made on cells obtained from biopsy samples. Tissue samples may be mechanically or enzymatically dis-aggregated to release individual living cells into a fluidic measurement platform. Functional measurements are then made on the living cells. In fact, after measuring a property of a cell, the cell remains alive and is available for subsequent analyses.

Instruments of the disclosure are used to measure cellular functions characteristic of cancer cells. The instruments are used to measure the growth of the cells by measuring mass or change in mass in the cells. Healthy, differentiated somatic cells from a tissue sample are expected to exhibit stable mass. When live cells from a tissue sample are measured with instruments of the disclosure, the detection of mass accumulation, or growth, indicates the presence of cancerous or pre-cancerous cells in the tissue. Moreover, known cancer cells that are responding favorably to a therapeutic may exhibit loss of mass. Instruments of the disclosure make sensitive and precise measurements of mass or change in mass indicative of cancer through the use of a suspended microchannel resonator. The instruments use a structure such as a cantilever that contains a fluidic microchannel. Living cells are flowed through the structure, which is resonated and its frequency of resonation is measured. The frequency at which a structure resonates is dependent on its mass and by measuring the frequency of at which the cantilever resonates, the instrument can compute a mass, or change in mass, of a living cell in the fluidic microchannel. By flowing the isolated living cells from the tissue sample through such devices, one observes the function of those cells, such as whether they are growing and accumulating mass or not. The mass accumulation or rate of mass accumulation can be related to clinically important property such as the presence of cancer cells or the efficacy of a therapeutic on cancer cells.

Thus, the disclosure provides for the rapid and precise measurement of functional properties of living cells in a tissue sample. Those functional properties provide a valuable marker of cancer activity. Significantly, the measurements may be made from a tissue sample and the measured cells may be living cancer cells from a solid tumor. Once the measurements are made, those living cells are available for further study, such as genome sequencing or other measurements. Thus the disclosure provides for the functional analysis of cancer cells.

Methods and instruments of the disclosure use a mass measurement technology to make use of a new type of biomarker with utility in precision medicine and oncology. Embodiments of the invention use microchannel resonators to precisely measure mass and mass changes in individual living cells. When used with cancer cells, those changes provide a functional, universal biomarker by which oncologists may monitor the progression of a cancer and determine how cancer cells respond to therapies. The resonators may be provided within a measurement instrument such as a bench-top instrument into which cells and samples are loaded. For cancers, various techniques may be used to load isolated living cells into a fluidic environment that makes a cell available to the microchannel resonator device. For example, methods and devices of the disclosure may be used to study living cells from solid tumors, wherein samples may be provided by biopsy or final needle aspiration.

In certain aspects, the invention provides a method for assessing a cancer biomarker. Such methods include obtaining a tissue sample comprising living cells, disaggregating the tissue sample and loading individual live cells into an input channel of a measurement instrument, and flowing the live cells through the measurement instrument to measure a functional property of the live cells. The tissue sample may be obtained from a human subject and the living cells may include cancer cells or immune cells. The functional property may be mass or mass accumulation rate (MAR). Preferably the mass is measured with a precision of at least about 0.01% of cell mass. The MAR may be measured with a precision of at least about 0.1% per hour. A duration of measuring the MAR is from about 20 minutes to about 3 hours. The functional property may be measured from the live cells within less than about three hours of the tissue sample being obtained by biopsy. The functional property may be measured from the live cells within less than about 3 hours after the disaggregating step.

In certain embodiments, the measurement instrument comprises a suspended microchannel resonator (SMR). In some embodiments, the live cells are measured and leave the instrument in a living state, accessible for a subsequent assay. For example, the method may include performing the assay on at least one cell from the living cells. The assay may be for example genome sequencing.

In certain preferred embodiments, the living cells are cancer cells. The tissue sample may be obtained from a solid tumor (such as a tumor of the bone, bladder, brain, breast, colon, esophagus, gastrointestinal tract, urinary tract, kidney, liver, lung, nervous system, ovary, pancreas, prostate, retina, skin, stomach, testicles, or uterus of a subject). The tissue sample may be provided via, for example, a fine needle aspirate, from a pleural effusion in a subject, or obtained from ascetic fluid in a subject. Masses or clumps of the tissue sample may be disaggregated to release individual cells into media such as culture or maintenance media. Disaggregating the cells may include physical, mechanical, chemical, or proteolytic disaggregation or a combination thereof. Thus in a preferred embodiment, a solid tumor is interrogated via fine needle aspiration to retrieve a cell mass, or tissue sample, that includes cancer cells. The cell mass may be deposited, e.g., on a nitrocellulose membrane and disaggregated using, e.g., proteases such as collagenase and/or displace. Live cells may be washed into a fluidic tube or system with and supported by a suitable media such as a Ham's nutrient mixture. The live cells may be flowed into a loading chamber of a measurement instrument such as a mass-measuring instrument that uses an SMR. A fluidic system of the instrument flows media through the SMR and controls pressure such that isolated individual living cancer cells flow through the SMR. Flow time through the SMR can be less than about 20 minutes to make a measurement of mass or mass accumulation rate and the measurement may be made within about 3 hours of obtaining the sample from the patient by FNA. A cell's mass or mass accumulation rate is measured in the SMR and the cell leaves the SMR alive and intact. The living cell can be passed through other SMRs or back through the same one. That living cell can also be sequestered in a collection tube and/or passed to another instrument for a downstream analysis such as nucleic acid capture and sequencing.

The method includes disaggregating the tissue sample and loading individual live cells into an input channel of a measurement instrument. Any suitable technique may be used for disaggregating the cells. For example, disaggregation may be mechanical, chemical, or enzymatic. A tissue sample or clump of cells may be digested with one more proteases to digest extracellular matrix among the cells, and the sample or clump may be washed with a maintenance or nutrient medium to load isolated individual living cells into a fluidic system such as a channel in a microfluidic device.

### BRIEF DESCRIPTION OF THE DRAWINGS

FIG. 1 diagrams steps of a method for measuring a cancer marker.

FIG. 2 shows obtaining a sample.

FIG. 3 shows loading live cells into an instrument for a functional measurement.

FIG. 4 diagrams a suspended microchannel resonator (SMR).

FIG. 5 shows a serial suspended microchannel resonator (sSMR) array.

FIG. 6 shows instruments for making a series of measurements from one living cell.

FIG. 7 shows a report as may be provided.

FIG. 8 shows a system useful for performing methods of the disclosure.

### DETAILED DESCRIPTION

The disclosure provides a mass measurement technology to make use of a new type of biomarker with utility in precision medicine and oncology. Embodiments of the technology use microchannel resonators to precisely measure mass and mass changes in individual living cells. When used with cancer cells, those changes provide a functional, universal biomarker by which oncologists may monitor the progression of a cancer and determine how cancer cells respond to therapies. The resonators may be provided within a measurement instrument such as a bench-top instrument into which cells and samples are loaded. For cancers, various techniques may be used to load isolated living cells into a fluidic environment that makes the cell available to the microchannel resonator device. For example, methods and devices of the disclosure may be used to study living cells from solid tumors, wherein samples may be provided by biopsy or final needle aspiration. Fine needle aspiration also provides the ability to sample from pleural effusions and ascites. Methods of the disclosure provide for getting isolated, living cancer cells into an environment that can service the microchannel resonator device. Methods for obtaining a cancer cell sample include accessing a tumor in a patient via fine needle aspirate to take a sample comprising cancer cells, disaggregating the sample to isolate at least one living cell in media, and providing the isolated, living cell to an analytical instrument for measuring a cancer marker. The methods may be used to address tumors or cancers of any suitable type. The instrument preferably makes a functional measurement in that it measures an aspect of cellular function e.g., a dynamic function such as growth, metabolism, synthesis of biological macromolecules, or similar. In certain embodiments, the functional measurement relates to cellular growth and may in particular be a measure of mass, change in mass, or rate of change in mass.

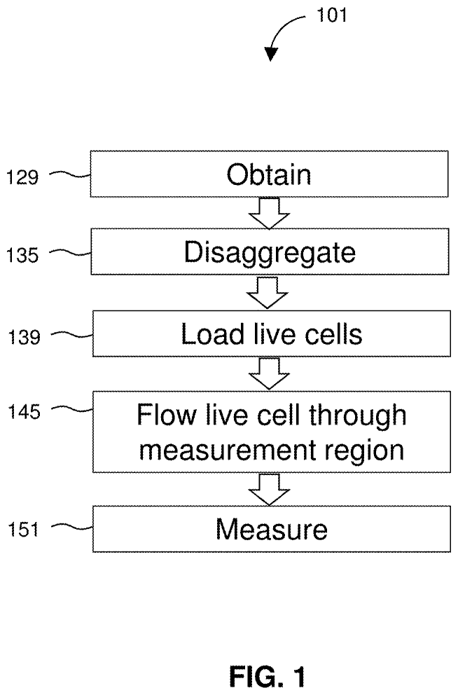

FIG. 1 diagrams steps of a method 101 for measuring 113 a cancer marker. The method 101 includes obtaining 129 a tissue sample comprising living cells. Tissue or clumps within the sample are disaggregated 135. With disaggregating 135 the tissue sample, the method 101 includes loading 139 individual live cells into an input channel of a measurement instrument. The instrument is an instrument suitable for making a functional, as compared to genomic, measurement of living cells. The method 101 includes flowing 145 the live cells through the measurement instrument to measure a functional property of the live cells. Specifically, individual live cells are flowed 145 through a measurement region of the instrument and the method can include measuring 151 a functional property of an individual living cell. In certain embodiments, the functional property comprises mass or mass accumulation rate (MAR).

Where the instrument measures mass or MAR, devices of the disclosure are provided that are capable of measuring the mass or MAR within certain valuable sensitivities or times. For example, mass measurement instruments that use a suspended microchannel resonator (SMR) are capable of measuring mass, mass change, or MAR with a precision of at least about 0.01% of a cell mass. SMR-based instruments are capable of measuring mass, mass change, or MAR with a precision of at least about 0.1% per hour. SMR-based instruments are capable of measuring mass, mass change, or MAR within a duration of measuring the MAR that is within about 20 minutes to about 3 hours.

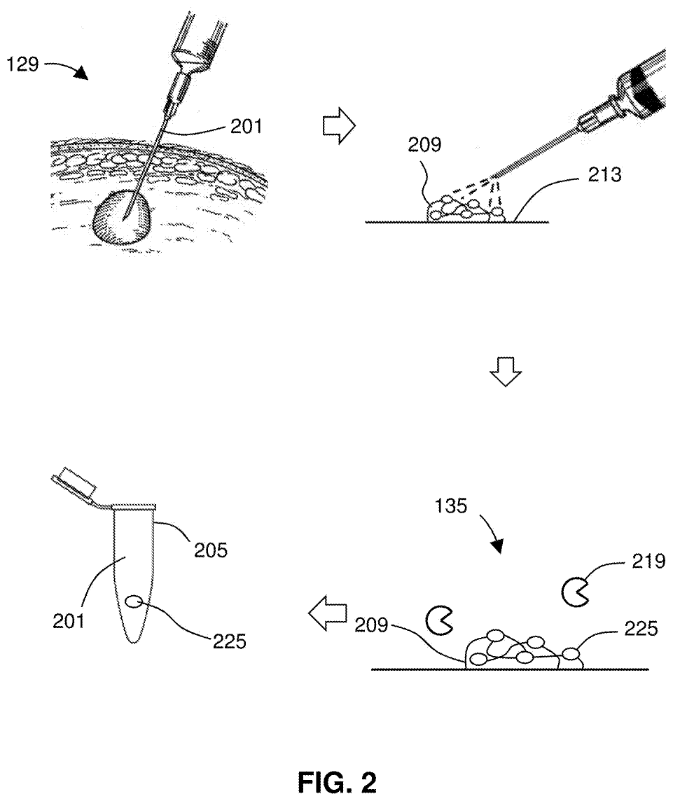

FIG. 2 shows obtaining 129 a sample 201. The sample 201 includes one or more live cells such as a cancer cell or an immune cell. Samples may be collected and stored in their own container 205, such as a tube or flask such as the 1.5 mL micro-centrifuge tube sold under the trademark EPPENDORF FLEX-TUBES by Eppendorf, Inc. (Enfield, Conn.). The one or more live cells are isolated from a biological sample of a patient known to have, or suspected of having, cancer. A biological sample may include a human tissue or bodily fluid and may be collected in any clinically acceptable manner. For example, the sample may include a fine needle aspirate or a biopsy from a tissue known to be, or suspected of being, cancerous. The sample may include a bodily fluid from a patient either known to include, or suspected of including, cancer cells or cancer-related cells (i.e., immune cells).

A tissue sample may include a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues.

A body fluid may be a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. A sample also may be media containing cells or biological material. A sample may also be a blood clot, for example, a blood clot that has been obtained from whole blood after the serum has been removed. In certain embodiments, the sample is blood, saliva, or semen collected from the subject.

Any suitable sample may be obtained. For example, the sample may include immune cells or cancer cells. The sample may include tissue of any type including healthy tissue or bodily fluid of any type. In some embodiments, the tissue sample is obtained from a pleural effusion in a subject. A pleural effusion is excess fluid that accumulates in the pleural cavity, the fluid-filled space that surrounds the lungs. This excess fluid can impair breathing by limiting the expansion of the lungs. Various kinds of pleural effusion, depending on the nature of the fluid and what caused its entry into the pleural space, may be sampled. A pneumothorax is the accumulation of air in the pleural space, and is commonly called a “collapsed lung”. In certain embodiments, the tissue sample is obtained from ascetic fluid in a subject. Ascites is the accumulation of fluid (usually serous fluid which is a pale yellow and clear fluid) that accumulates in the abdominal cavity. The abdominal cavity is located below the chest cavity, separated from it by the diaphragm. The accumulated fluid can have many sources such as liver disease, cancers, congestive heart failure, or kidney failure. The most common cause of ascites is advanced liver disease and methods 101 of the disclosure may be used for monitoring liver disease.

In some embodiments, the sample is from a patient having or suspected of having a cancer. The cancer may be a leukemia, a lymphoma, or a myeloma. The cancer may be a melanoma, a carcinoma, or a sarcoma. In certain embodiments, the cancer involves a solid tumor of, for example, the esophagus, kidneys, uterus, ovaries, thyroid, breast, liver, gallbladder, stomach, pancreas, or colon. A sample may be obtained 129 by a biopsy procedure.

In certain preferred embodiments, the tissue sample is obtained from a solid tumor and the tumor is from one selected from the group consisting of a bone, bladder, brain, breast, colon, esophagus, gastrointestinal tract, urinary tract, kidney, liver, lung, nervous system, ovary, pancreas, prostate, retina, skin, stomach, testicles, and uterus of a subject. Fine needle aspiration may be used to obtain 129 the sample from a tumor. As shown, a solid tumor is interrogated via fine needle aspiration to retrieve a cell mass, or tissue sample, that includes cancer cells. Methods may include using a needle 201, preferably fine-needle aspiration biopsy using a sharp 25-gauge, 1-inch long needle. One or more separate aspirates may be made from different areas of the tumor. A suitable needle 201 is the sharp 25-gauge, 1-inch long needle sold under the trademark PRECISION GLUIDE by BD (Franklin Lakes, N.J.). The needle may be attached via flexible plastic tubing to a 10 ml aspirating syringe. The procedure may be monitored under indirect ophthalmoscopy.

The biopsy needle may be passed into a lesion or tumor. Once the tip of the needle is advanced into the lesion, the tumor cells are aspirated. The plunger of the syringe may be forcibly pulled and quickly released a few times, allowing the suction force in the line to equilibrate. The needle is withdrawn and a tissue sample or clump of cells is deposited in or on a substrate 213. Any suitable substrate 213 may be used such as a slide, culture dish, membrane, or other material. In some embodiments, the clump of cells 209 is deposited on a surface within a collection tube or flask, such as a 1.5 mL microcentrifuge tube sold under the trademark EPPENDORF. Each aspirate may be flushed into the flask using culture media, saline, or a maintenance/nutrient media. The aspiration material may be filtered to deposit clumps or samples of tissue on the surface of a filter membrane. The cell mass may be deposited, e.g., on a nitrocellulose membrane and disaggregated using, e.g., proteases such as collagenase and/or displace. Live cells may be washed into a fluidic tube or system with and supported by a suitable media such as a Ham's nutrient mixture. For information, see Rajer, 2005, Quantitative analysis of fine needle aspiration biopsy samples, Radiol Oncol 39(4):269-72, incorporated by reference.

The tissue sample or clump of cells 209 is disaggregated 135. Any suitable technique may be used to disaggregate 135 the tissue sample/clump of cells 209. For example, disaggregation may include physical or mechanical disaggregation, chemical disaggregation, proteolytic disaggregation, or any combination thereof. In some embodiments, proteolytic disaggregation is performed using one or more enzymes 219. Any suitable enzymes may be used. In some embodiments, the tissue sample/clump of cells 209 is washed with and digested by collagenase I and dispase II. The resultant free cells may be held in a suitable nutrient media such as, for example, Ham's F12 Kaighn's Modification medium in presence of 1 mU/mL bovine thyrotropin (TSH), 10 μg/mL human insulin, 6 μg/mL transferrin, and 10-8 M hydrocortisone.

Thus the method 101 may include obtaining a fine needle aspirate tissue sample that includes live cancer cells that have been disaggregated from any tissue or clump so that individual live cells may be separately addressed, e.g., subjected to a measurement of some functional property of those cells. Other methods may be used for obtaining a sample 201 and isolating at least one living cell 225.

The isolation of a live cell 225 from the biological sample 201 may be performed via any known isolation techniques and methods for maintaining a viable collection of cells, which may include one or cancer and/or cancer-related immune cells (e.g., lymphocytes includes T-cells and/or B-cells). For example, if the sample is a tissue sample from a tumor or growth suspected of being cancerous, the tissue sample may undergo any known cell isolation, separation, or dissociation techniques which may involve physical methods (i.e., use of mechanical force to break apart cellular adhesions) and/or reagent-based methods (i.e., use of fluid mediums to break apart cellular adhesions). For example, in one embodiment, a tissue sample (i.e., a fine needle aspirate from a tumor) may be disaggregated to produce a suspension of individual live cells to allow for analysis of cells independently. The tissue sample may undergo initial disaggregation by way of application of a physical force alone to break the tissue sample into smaller pieces, at which point the sample may be exposed to proteolytic enzymes that digest cellular adhesion molecules and/or the underlying extracellular matrix to thereby provide single cells within a suspension. It should be noted that the reagents selected for assisting in the disaggregating step should keep the cells intact and not kill the cells.

Other methods currently used for single cell isolation include, but are not limited to, serial dilution, micromanipulation, laser capture microdissection, FACS, microfluidics, Dielectrophoretic digital sorting, manual picking, and Raman tweezers. Manual single cell picking is a method is where cells in a suspension are viewed under a microscope, and individually picked using a micropipette, while Raman tweezers is a technique where Raman spectroscopy is combined with optical tweezers, which uses a laser beam to trap, and manipulate cells. Dielectrophoretic (DEP) digital sorting method utilizes a semiconductor controlled array of electrodes in a microfluidic chip to trap single cells in DEP cages, where cell identification is ensured by the combination of fluorescent markers with image observation and delivery is ensured by the semiconductor controlled motion of DEP cages in the flow cell.

Using instruments and methods of the disclosure, a functional property may be measured from the live cells within less than about three to 48 hours of the tissue sample being obtained by biopsy. Instruments and methods of the disclosure are capable of making measurements within a few (e.g., 3) hours of a biopsy procedure. In practical use, the turnaround time may be 36 or 48 hours. The sample may optionally be held (e.g., in a media such a nutrient medium) for a period (e.g., overnight) in a controlled environment such as an incubator (e.g., at about 35 or 37 degrees C., i.e., physiological temperature). Notably, using the mass measurement instruments of the disclosure, the turnaround time from disaggregation to measurement result may be less than a couple of hours. The functional property may be measured from the live cells within less than about two or three hours after the disaggregating step.

The live cells may be flowed into a loading chamber of a measurement instrument such as a mass-measuring instrument that uses an SMR. A fluidic system of the instrument flows media through the SMR and controls pressure such that isolated individual living cancer cells flow through the SMR. Flow time through the SMR can be less than about 20 minutes to make a measurement of mass or mass accumulation rate and the measurement may be made within about three hours to about forty-eight hours of obtaining the sample from the patient by FNA. A cell's mass or mass accumulation rate is measured in the SMR and the cell leaves the SMR alive and intact. The living cell can be passed through other SMRs or back through the same one. That living cell can also be sequestered in a collection tube and/or pass to another instrument for a downstream analysis such as nucleic acid capture and sequencing.

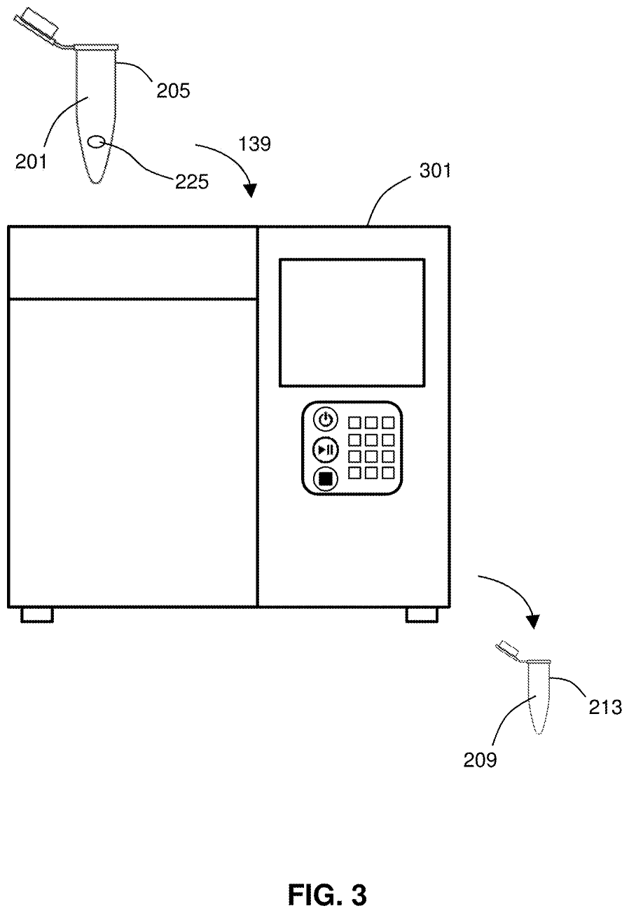

FIG. 3 shows loading 139 of the one or more live cells 225 into an instrument 301 capable of measuring 151 a functional property of the cells 225. The instrument 301 is operable to measure a functional cancer biomarker in the one or more live cells, such as single-cell biophysical properties, including, but not limited to, mass, growth rate, and mass accumulation of an individual living cell. The initial assay may generally be performed with an instrument 301 comprising a suspended microchannel resonator (SMR). The SMR may be used to precisely measure biophysical properties, such as mass and mass changes, of a single cell flowing therethrough. The mass change may be mass accumulation rate (MAR). When used with cancer cells, those changes provide a functional, universal biomarker by which medical professionals (e.g., oncologists) may monitor the progression of a cancer and determine how cancer cells respond to therapies.

The SMR may comprise an exquisitely sensitive scale that measures small changes in mass of a single cell. When cancer cells respond to cancer drugs, the cells begin the process of dying by changing mass within hours. The SMR can detect this minor weight change. That speed and sensitivity allow the SMR to detect a cancer cell's response to a cancer drug while the cell is still living. Upon flowing the live cells through the SMR, a functional biomarker, such as mass or MAR, in the one or more live cells is obtained. MAR measurements characterize heterogeneity in cell growth across cancer cell lines. Individual live cells are able to pass through the SMR, wherein each cell is weighed multiple times over a defined interval. The SMR includes multiple sensors that are fluidically connected, such as in series, and separated by delay channels. Such a design enables a stream of cells to flow through the SMR such that different sensors can concurrently weigh flowing cells in the stream, revealing single-cell MARs. The SMR is configured to provide real-time, high-throughput monitoring of mass change for the cells flowing therethrough. Therefore, the biophysical properties, including mass and/or mass changes (e.g., MAR), of a single cell can be measured. Such data can be stored and used in subsequent analysis steps, as will be described in greater detail herein. The specific function and details regarding instrument 301, including an exemplary suspended microchannel of an SMR, are described in greater detail herein.

Upon passing through the instrument 301, single cells remain viable and can be isolated downstream from the instrument 301 and are available to undergo the subsequent assays. As shown, a sample 209 of the one or more live cells having undergone the first assay (i.e., passing through the instrument 301) are collected in a suitable container 213 and are then available to undergo a second assay.

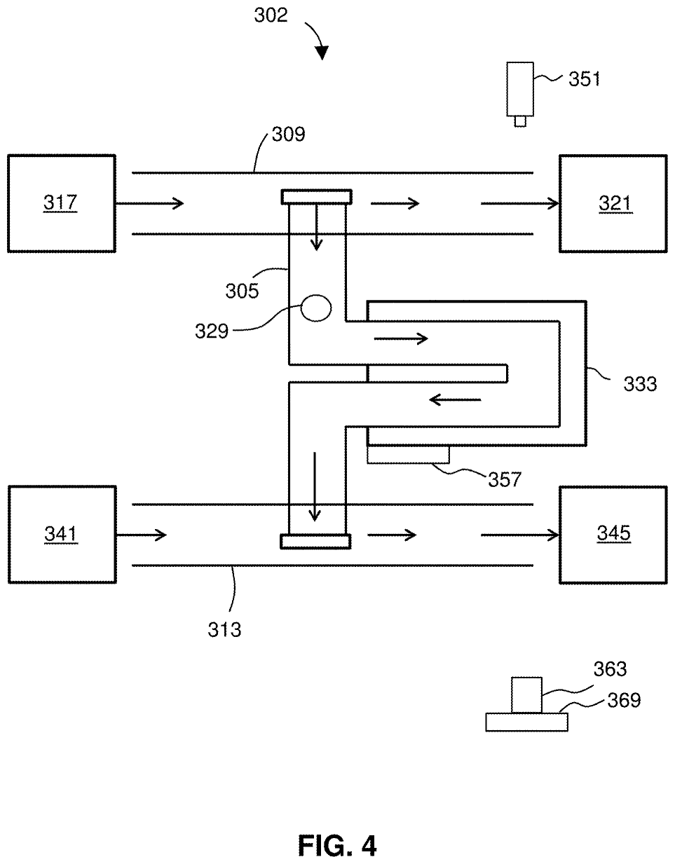

FIG. 4 shows a suspended microchannel resonator (SMR) device 302 of the disclosure. The SMR device 302 includes a microchannel 305 that runs through a cantilever 333, which is suspended between an upper bypass channel 309 and a lower bypass channel 313. Having the two bypass channels allows for decreased flow resistance and accommodates the flow rate through the microchannel 305. Sample eluate 317 flows through the upper bypass channel 309, wherein a portion of the eluate 317 collects in the upper bypass channel waste reservoir 321. The calibration method is being depicted. A reference material 329 with cell-like properties (and optionally a known mass) has been introduced 120 into the channel 305.

A portion of the eluate 317 including the reference material 329 flows through the suspended microchannel 305. The flow rate through the suspended microchannel 305 is determined by the pressure difference between its inlet and outlet. Since the flow cross section of the suspended microchannel is about 70 times smaller than that of the bypass channels, the linear flow rate can be much faster in the suspended microchannel than in the bypass channel, even though the pressure difference across the suspended microchannel is small. Therefore, at any given time, it is assumed that the SMR is measuring the eluate that is present at the inlet of the suspended microchannel. The sample includes a live cell or material with cell-like properties.

The reference material 329 flows through the suspended microchannel 305. The suspended microchannel 305 extends through a cantilever 333 which sits between a light source 351 and a photodetector 363 connected to a chip 369 such as a field programmable gate array (FPGA). The cantilever is operated on by an actuator, or resonator 357. The resonator 357 may be a piezo-ceramic actuator seated underneath the cantilever 333 for actuation. The cell 329 flows from the upper bypass channel 309 to the inlet of the suspended microchannel 305, through the suspended microchannel 305, and to the outlet of the suspended microchannel 305 toward the lower bypass channel 313. A buffer 341 flows through the lower bypass channel towards a lower bypass channel collection reservoir 345. After the reference material 329 is introduced to the lower bypass channel 313, the reference material 329 is collected in the lower bypass collection reservoir 345.

By flowing the reference material 329 through the SMR device 302, a reading or measurement may be made and the readout of the measurement may be adjusted to until it converges on the known mass of the reference material 329 to thereby calibrate the instrument 301. Once the instrument is thus calibrated, it may be used for making measurements of functional properties of live cells such as measurements of mass or mass accumulation rate (MAR). MAR measurements characterize heterogeneity in cell growth across cancer cell lines. Individual live cells are able to pass through the SMR, wherein each cell is weighed multiple times over a defined interval. The SMR includes multiple sensors that are fluidically connected, such as in series, and separated by delay channels. Such a design enables a stream of cells to flow through the SMR such that different sensors can concurrently weigh flowing cells in the stream, revealing single-cell MARs. The SMR device 302 provides real-time, high-throughput monitoring of mass change for the cells flowing therethrough. Therefore, the biophysical properties, including mass and/or mass changes (e.g., MAR), of a single cell can be measured. Such data can be stored and used in subsequent analysis steps, as will be described in greater detail herein. Various embodiments of SMR and sSMR instruments and methods of use include those manufactured by Innovative Micro Technology (Santa Barbara, Calif.) and described in U.S. Pat. Nos. 8,418,535 and 9,132,294, the contents of each of which are hereby incorporated by reference in their entirety.

Upon passing through the instrument 301, single cells remain viable and can be isolated downstream from the instrument 301 and are available to undergo the subsequent assays. As shown, a sample 209 of the one or more live cells having undergone the first assay (i.e., passing through the instrument 301) are collected in a suitable container 213 and are then available to undergo a second assay.

In some embodiments, the instrument 301 comprises an array of SMRs with a fluidic channel passing therethrough.

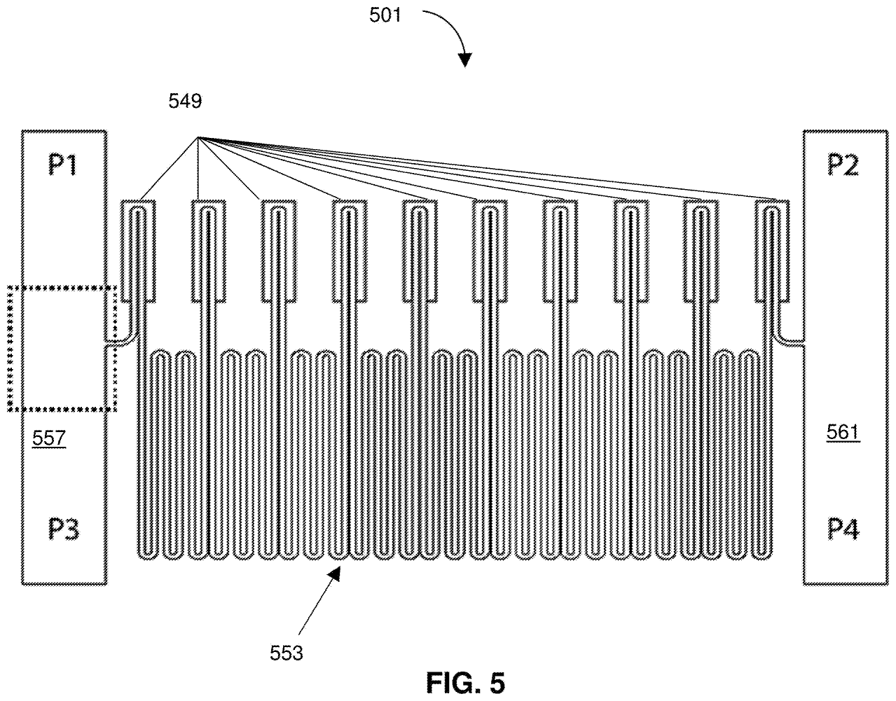

FIG. 5 shows a serial suspended microchannel resonator (sSMR) array 501. Instruments 301 may include one or more sSMR array 501 to make reliably sensitive and precise measurements of mass or change in mass. Instead of a single suspended microchannel, the instrument 301 may include an sSMR array 501, which includes a plurality of cantilevers 549 and a plurality of delay channels 553. A live, malignant cancer cell flows from the first bypass channel 557 through the cantilevers 549 and delay channels 553 to the second bypass channel 561. Pressure differences in the first bypass channel 557 are indicated by P1 and P2, and pressure differences in the second bypass channel 561 are indicated by P3 and P4. Living cells are flowed through the sSMR array 501, which is resonated and its frequency of resonation is measured. The frequency at which a structure resonates is dependent on its mass and by measuring the frequency of at which the cantilever resonates, the instrument can compute a mass, or change in mass, of a living cell in the fluidic microchannel. By flowing the isolated living cells from the tissue sample through such devices, one may observe the functions of those cells, such as whether they are growing and accumulating mass or not. The mass accumulation or rate of mass accumulation can be related to clinically important property such as the presence of cancer cells or the efficacy of a therapeutic on cancer cells.

Methods for measuring single-cell growth are based on resonating micromechanical structures. The methods exploit the fact that a micromechanical resonator's natural frequency depends on its mass. Adding cells to a resonator alters the resonator's mass and causes a measurable change in resonant frequency. Suspended microchannel resonators (SMRs) include a sealed microfluidic channel that runs through the interior of a cantilever resonator. The cantilever itself may be housed in an on-chip vacuum cavity, reducing damping and improving frequency (and thus mass) resolution. As a cell in suspension flows through the interior of the cantilever, it transiently changes the resonant frequency of the cantilever in proportion to the buoyant mass of the cell. SMRs weigh single mammalian cells with a resolution of 0.05 pg (0.1% of a cell's buoyant mass) or better. The sSMR array 501 includes an array of SMRs fluidically connected in series and separated by delay channels between each cantilever 549. The delay channels give the cell time to grow as it flows between cantilevers.

Devices may be fabricated as described in Lee, 2011, Suspended microchannel resonators, Lab Chip 11:645 and/or Burg, 2007, Weighing of biomolecules, Nature 446:1066-1069, both incorporated by reference. Large-channel devices (e.g., useful for PBMC measurements) may have cantilever interior channels of 15 by 20 μm in cross-section, and delay channels 20 by 30 μm in cross-section. Small-channel devices (useful for a wide variety of cell types) may have cantilever channels 3 by 5 μm in cross-section, and delay channels 4 by 15 μm in cross-section. The tips of the cantilevers 549 in the sSMR array 501 may be aligned so that a single line-shaped laser beam can be used for optical-lever readout. The cantilevers may be arrayed such that the shortest (and therefore most sensitive) cantilevers are at the ends of the array. Before use, the sSMR array 501 may be cleaned with piranha (3:1 sulfuric acid to 50% hydrogen peroxide) and the channel walls may be passivated with polyethylene glycol (PEG) grafted onto poly-L-lysine. In some embodiments, a piezo-ceramic actuator seated underneath the device is used for actuation. The instrument 301 may include low-noise photodetector, Wheatstone bridge-based amplifier (for piezo-resistor readout), and high-current piezo-ceramic driver. To avoid the effects of optical interference between signals from different cantilevers (producing harmonics at the difference frequency), the instrument may include a low-coherence-length light source (675 nm super-luminescent diode, 7 nm full-width half maximum spectral width) as an optical lever. After the custom photodetector converts the optical signal to a voltage signal, that signal is fed into an FPGA board, in which an FPGA implements twelve parallel second-order phase-locked loops which each both demodulate and drive a single cantilever. The FPGA may be a Cyclone IV FPGA on a DE2-115 development board operating on a 100 MHz clock with I/O provided via a high-speed AD/DA card operating 14-bit analog-to-digital and digital-to-analog converters at 100 MHz.

To operate all cantilevers 549 in the sSMR array 501, the resonator array transfer function is first measured by sweeping the driving frequency and recording the amplitude and phase of the array response. Parameters for each phase-locked loop (PLL) are calculated such that each cantilever-PLL feedback loop has a 50 or 100 Hz FM-signal bandwidth. The phase-delay for each PLL may be adjusted to maximize the cantilever vibration amplitude. The FM-signal transfer function may be measured for each cantilever-PLL feedback loop to confirm sufficient measurement bandwidth (in case of errors in setting the parameters). That transfer function relates the measured cantilever-PLL oscillation frequency to a cantilever's time-dependent intrinsic resonant frequency. Frequency data for each cantilever may be collected at 500 Hz, and may be transmitted from the FPGA to a computer. The device may be placed on a copper heat sink/source connected to a heated water bath, maintained at 37 degrees C. The sample is loaded into the device from vials pressurized under air or air with 5% CO2 through 0.009 inch inner-diameter fluorinated ethylene propylene (FEP) tubing. The pressurized vials may be seated in a temperature-controlled sample-holder throughout the measurement. FEP tubing allows the device to be flushed with piranha solution for cleaning, as piranha will damage most non-fluorinated plastics. To measure a sample of cells, the sSMR array 501 may initially flushed with filtered media, and then the sample may be flushed into one bypass channel. On large-channel devices, between one and two psi may be applied across the entire array, yielding flow rates on the order of 0.5 nL/s (the array's calculated fluidic resistance is approximately 3 10\hat{ }16 Pa/(m3/s). For small-channel devices, 4-5 psi may be applied across the array, yielding flow rates around 0.1 nL/s. Additionally, every several minutes new sample may be flushed into the input bypass channel to prevent particles and cells from settling in the tubing and device. Between experiments, devices may be cleaned with filtered 10% bleach or piranha solution.

For the data analysis, the recorded frequency signals from each cantilever 449 are rescaled by applying a rough correction for the different sensitivities of the cantilevers. Cantilevers differing in only their lengths should have mass sensitivities proportional to their resonant frequencies to the power three-halves. Therefore each frequency signal is divided by its carrier frequency to the power three-halves such that the signals are of similar magnitude. To detect peaks, the data are filtered with a low pass filter, followed by a nonlinear high pass filter (subtracting the results of a moving quantile filter from the data). Peak locations are found as local minima that occur below a user-defined threshold. After finding the peak locations, the peak heights may be estimated by fitting the surrounding baseline signal (to account for a possible slope in the baseline that was not rejected by the high pass filter), fitting the region surrounding the local minima with a fourth-order polynomial, and finding the maximum difference between the predicted baseline and the local minima polynomial fit. Identifying the peaks corresponding to calibration particles allows one to estimate the mass sensitivity for each cantilever, such that the modal mass for the particles is equal to the expected modal mass. Peaks at different cantilevers 549 that originate from the same cell are matched up to extract single-cell growth information. The sSMR array 501 and can measure live cells.

Various embodiments of SMR and sSMR instruments, as well as methods of use, include those instruments/devices manufactured by Innovative Micro Technology (Santa Barbara, Calif.) and described in U.S. Pat. Nos. 8,418,535 and 9,132,294, all incorporated by reference. Notably, when a measurement is made using an instrument 301 of the disclosure, the live cells are measured and leave the instrument 301 in a living state, accessible for a subsequent assay.

Thus, methods of the present invention allow for multiple assays to be performed on a sample of live cells, thereby providing real-time morphological and phenotypic insight of such cells. In particular, the initial assay is performed on an instrument 301 including a suspended microchannel resonator (SMR), which has an exquisitely sensitive scale that can measure small changes in mass of a single cell. When cancer cells respond to cancer drugs, such cells start their process of dying by changing mass within hours. The speed and sensitivity of the SMR enable the SMR to detect a cancer cell's response (i.e., changes in mass) to therapies while the cell is still living, wherein such responses are not discernable via genomic measurements and can only be obtained on live cells. The cancer cell's responses provide a functional, universal biomarker by which medical professionals may monitor the progression of a cancer and determine how cancer cells respond to therapies. In particular, the biophysical properties (i.e., mass, change in mass, and MAR) offer unique insights into a wide range of biological phenomena of a live cancer cell, including, but not limited to, basic patterns of single-cell mass and growth regulation, biophysical changes associated with immune cell activation, and cancer cell heterogeneity in the presence or absence of a drug therapy.

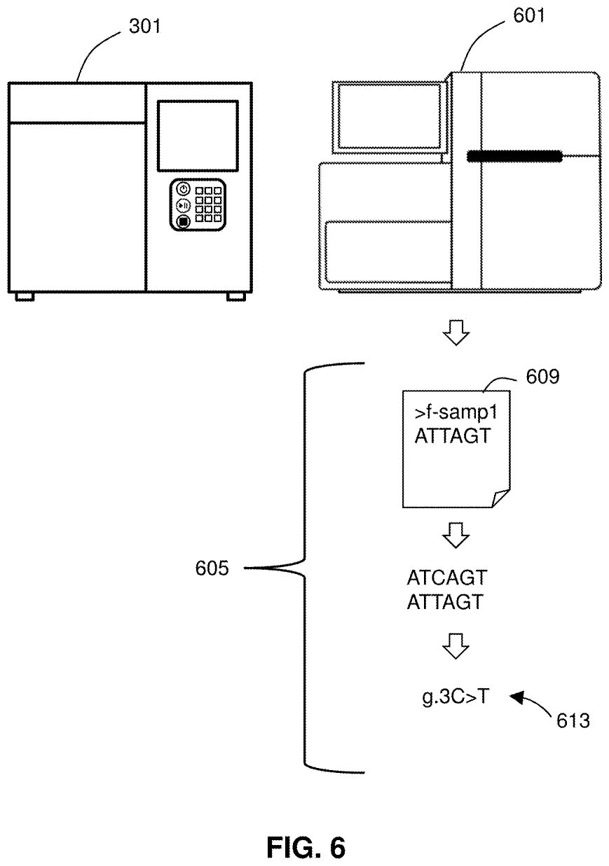

FIG. 6 illustrates a functional measurement instrument 301 and a second instrument 601 that may be employed to make a measurement on a cell that has been measured in the functional measurement instrument 301. In some embodiments, the second instrument 601 is a nucleic acid sequencer such as one of the next-generation sequencing (NGS) instruments sold under the trademarks MISEQ or HISEQ by Illumina, Inc. Where the second instrument 601 is an NGS instrument, methods of the disclosure include sequencing nucleic acid from a cell 225 from the sample 201 to obtain genetic information from the sample.

Sequencing generally includes isolating and optionally amplifying the nucleic acid from the cell 225, operating the instrument 601 to sequence the nucleic acid and produce sequence reads 609, and analyzing 605 the sequence reads to obtain genetic information 613 from the cell 225 from the patient. Genetic information may include the identification of genetic mutations (i.e., by variant calling) or chromosomal alternations which include sub-chromosomal copy number variation (CNV) and/or aneuploidy (e.g., such as trisomy). In certain embodiments, genome sequencing or whole exome sequence is performed, or a panel of genes is sequenced, to determine the presence or identity of a set, or “panel” of cancer-related mutations such as single-nucleotide polymorphisms (SNPs). SNPs may be identified by variant calling, which typically involves mapping sequence reads to a reference and reporting differences as variants. Sequence analysis can include any combination of read assembly or mapping. See U.S. Pat. No. 8,209,130, incorporated by reference, for discussion of sequencing and sequence analysis.

Using a functional measurement instrument 301 and a second instrument 601 such as a genome sequencer, the disclosure provides methods for evaluating disease, such as cancer, by performing multiple assays on one single live cell. The living cell may be isolated from a sample from a patient, wherein the sample is either known to have, or suspecting of having, cancer cells or cancer-related cells (e.g., immune cells). The data obtained from the multiple assays may be analyzed and linked to thereby provide a characterization of any given cell having undergone analysis, which, in turn, allows for evaluation of the sample either known to be, or suspected of being, cancerous. A report may be generated based on the data analysis, wherein the report provides information related to the cancer evaluation, including, but not limited to, whether the sample tested positive for cancer, a determination of a stage or progression of cancer, and a customized treatment plan tailored to an individual patient's cancer diagnosis.

The single cell 225 remains viable upon passing through the SMR instrument 301 and can further be isolated downstream from the instrument where cells may undergo subsequent assays to obtain additional measurements of the one or more live cells, such as genetic data. As such, data from the additional assays can be analyzed with data from the initial assay, to thereby provide a detailed characterization of any given cell, in turn allowing for a more comprehensive cancer evaluation of the patient sample. A report may be generated based on the data analysis, wherein the report provides information related to the cancer evaluation, including, but not limited to, whether the sample tested positive for cancer, a determination of a stage or progression of cancer, and a customized treatment plan tailored to an individual patient's cancer diagnosis. As such, the methods of the present invention can improve outcomes of cancer treatment, avoid any unnecessary cancer treatment, and reduce overall healthcare costs.

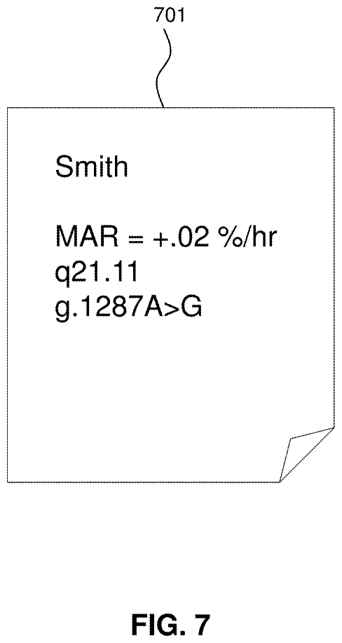

FIG. 7 shows a report 701 as may be provided. The report 701 may include any suitable patient information including identity along with information related to the cancer evaluation, including, but not limited to, whether the sample tested positive for cancer, a determination of a stage or progression of cancer, and a customized treatment plan tailored to an individual patient's cancer diagnosis. In some embodiments, the report 701 describes one or more genetic sequence alterations and the measured functional biomarker (i.e., mass, change in mass, mass accumulation rate, etc.) in the live cells from the patient. The report 701 may be anonymized (e.g., according to an encoded patient ID). The report 701 may be provided on paper or may be stored or transmitted electronically, e.g., as a PDF or XML document. The report may include any clinically-significant biophysical properties (e.g., mass, change in mass, mass accumulation rate (MAR)) and/or genetic information determined from the isolated cells. For patient reporting or notification, systems and methods of the invention may be used to retrieve medical/clinical information from an outside database. The outside database may be a clinical decision support system such as UP2DATE by Wolters-Kluwer. Any suitable clinical decision support resources may be included in the outside database that is queried by the system. Other suitable resources include the medical reference resource sold under the name EPOCRATES by Athena Health (Watertown, Mass.). Other clinical decision support (CDS) resources that may be accessed may include the PREDICT (Pharmacogenomic Resource for Enhanced Decisions in Care and Treatment) project, the CLIPMERGE (Clinical Implementation of Personalized Medicine through Electronic Health Records and Genomics) program, and the SMART (Substitutable Medical Apps Reusable Technologies) Genomics Adviser. Thus the method 101 may include analyzing the sequence data and the measured functional biomarker to determine a stage or progression of the cancer.

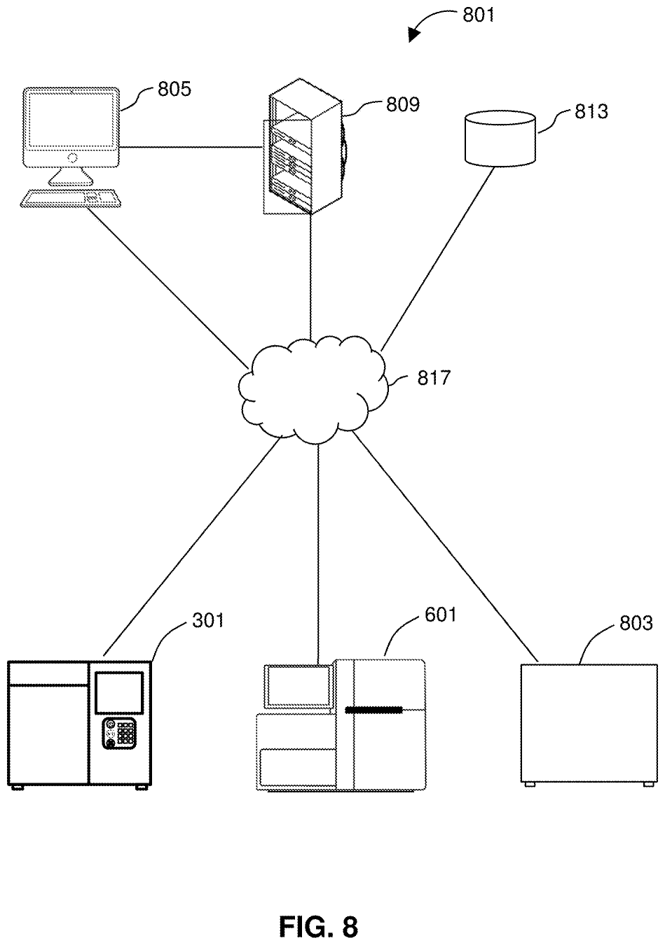

FIG. 8 shows a system 801 useful for performing methods of the disclosure. The preferably includes an SMR instrument 301 and at least one computer 805. The system 801 may optionally also include any one or more of a server 809, storage 813, a sequencing instrument 601, and any additional analysis instruments 803 for performing additional assays on the one or more cells downstream of the initial assay performed by instrument 301. Any of those elements may interoperate via a network 817. Any one of the instruments may include its own on-board computer. The computer 805 may include one or more processors and memory as well as an input/output mechanism. Where methods of the invention employ a client/server architecture, steps of methods of the invention may be performed using the server 809, which includes one or more of processors and memory, capable of obtaining data, instructions, etc., or providing results via an interface module or providing results as a file. The server 809 may be provided by a single or multiple computer devices, such as the rack-mounted computers sold under the trademark BLADE by Hitachi. The server 809 may be provided as a set of servers located on or off-site or both. The server 809 may be owned or provided as a service. The server 809 or the storage 813 may be provided wholly or in-part as a cloud-based resources such as Amazon Web Services or Google. The inclusion of cloud resources may be beneficial as the available hardware scales up and down immediately with demand. The actual processors—the specific silicon chips—performing a computation task can change arbitrarily as information processing scales up or down. In an embodiment, the server 809 includes one or a plurality of local units working in conjunction with a cloud resource (where local means not-cloud and includes or off-site). The server 809 may be engaged over the network 817 by the computer 805.

In system 801, each computer preferably includes at least one processor coupled to a memory and at least one input/output (I/O) mechanism. A processor will generally include a chip, such as a single core or multi-core chip, to provide a central processing unit (CPU). A processor may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Generally, each computer includes a non-transitory memory such as a solid state drive, flash drive, disk drive, hard drive, etc. While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

The system 701 or components of system 701 may be used to perform methods described herein. Instructions for any method step may be stored in memory and a processor may execute those instructions.

The system 701 thus includes at least one computer (and optionally one or more instruments) operable to obtain one or more live cells isolated from a sample of a patient, wherein the one or more live cells comprise at least one of a cancer cell and a cancer-related immune cell. The system 701 is further operable to perform a first assay on the one or more live cells, wherein the first assay comprises measuring a functional cancer biomarker in the one or more live cells. The system 701 is optionally further operable to perform a second assay on the one or more live cells having undergone the first assay. The system 701 is further operable to analyze data from the second assay and the measured cancer biomarker to determine at least a stage or progression of the cancer. Using the computer 701, the system is operable to provide a report comprising any suitable patient information including identity along with information related to the cancer evaluation, including, but not limited to, specific data associated with the first and second assays, a determination of a stage or progression of cancer, and personalized treatment tailored to an individual patient's cancer.

The invention provides systems and methods for measuring how living cells function. The measurements can be made from tissue biopsy samples to measure functional properties of living cells from a solid tumor. After measuring a functional property of a cell, the cell remains alive and is available for other subsequent analyses. A system of the disclosure may be used to perform a method for measuring a cancer marker. The method implemented by the system includes obtaining a tissue sample comprising living cells, disaggregating the tissue sample and loading individual live cells into an input channel of a measurement instrument, and flowing the live cells through the measurement instrument to measure a functional property of the live cells.

The invention provides methods and devices that are used to rapidly measure functional properties of individual living cells from a patient. With minimal processing and no requirement of overnight culturing, isolated living cells are obtained from blood draws or tissue samples. Instruments of the invention measure functional properties of those living cells, including important properties such as cellular growth or health. The measurements are rapid and may be performed within less than a few hours of sample collection from a patient. The measurements require as little of ten or twenty minutes of instrument run time, and obtain measurement precision of at least 0.01%. Upon completion of the measurements, the individual cells are still live and viable and can be subject to further measurements such as passage back through the same instrument later, to detect changes over time, or genomic analysis, to correlate genetic markers to cellular viability.

Methods and devices of the invention add a new modality of measurement that is orthogonal to genomics. The functional measurements are rapid and free of incubation requirements: living cells are liberated from patient samples and flowed directly into an instrument. Functional properties such mass changes measured in those cells can reveal, for example, if the cells are growing, stationary, or atrophying. Those features of cellular life may be hallmarks of health, cancer, or drug response, and thus methods and devices of the disclosure are valuable tools for precision medicine. Precision Medicine refers to the tailoring of medical treatment to individual characteristics of a patient and the ability to classify individuals into subpopulations that differ in their susceptibility to a particular disease or treatment. Precision medicine often involves genomic or molecular analysis of an individual patient's disease at the molecular level and the selection of targeted treatments to address that individual patient's disease process. In theory, therapeutic interventions are concentrated on those who will benefit, sparing expense and side effects for those who will not. Historically, next-generation sequencing (NGS) technologies make up the core of precision medicine. Clinicians use NGS technologies to screen for cancer-associated mutations or to study gene expression levels. Now, when coupled with existing approaches based on next-generation sequencing, functional measurements according to the invention provide for multi-dimensional precision medicine with benefits in disease areas such as oncology.

Methods and devices of the invention may be used to identify malignant cancer cells in a blood or tissue sample from a patient. Those tools may also be used as an ex vivo test of drug response, useful for therapeutic selection. Moreover, after treatment of a patient, methods and devices of the invention may be used to monitor recurrence, remission, or relapse. Thus the invention provides for the improvement of patient care, greater chances of successful cancer treatment, and increased patient life spans.

Methods and instruments of the disclosure are useful for precisely and rapidly measuring growth rates of living individual cells using a small amount of a sample. In the invention, a small amount of a sample is used to observe and measure a single cell, as opposed to observing a population of cells in traditional methods. Therefore, a small amount of cells can be obtained directly from a subject, suspended in media, and then introduced to a measurement instrument without the need to add additional time-consuming steps, such as culturing the cells. In the invention, the cells from the biological sample are separated when flowing through a microfluidic channel of the measurement instrument and the growth rate of individual cells is measured.

The invention requires a small sample size compared to sample sizes necessary in other measurement methods. For example, in the present invention, the sample comprises about 500 or fewer cells. A small amount of cells may be used because of the precision of the methods of measurement of the present invention. Therefore, methods of the invention may be used when limited tissue samples are available for testing and measurement. For example, a tissue sample may comprise about 10,000 cells. Such a tissue sample does not have enough cells present in the sample for traditional measurement methods, such as optics measurement methods. However, methods of the invention may use a sample having about 500 cells to measure mass accumulation rate (MAR). If a sample of about 10,000 cells is provided, methods of the present invention may use that sample and test 20 different conditions. For example, 500 cells may be dosed with a first drug to determine the effects of the drug on mass accumulation rate of the cells. Therefore, as many as 20 different drugs may be tested with a sample containing 10,000 cells.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for measuring a cancer marker, the method comprising:
obtaining a tissue sample comprising living cells;
disaggregating the tissue sample and loading individual live cells into an input channel of a measurement instrument; and
flowing the live cells through the measurement instrument to measure a functional property of the live cells.

2. The method of claim 1, wherein the tissue sample is obtained from a human subject and the living cells include cancer cells or immune cells.

3. The method of claim 1, wherein the live cells are measured and leave the instrument in a living state, accessible for a subsequent assay.

4. The method of claim 3, further comprising performing the assay on at least one cell from the living cells,

5. The method of claim 4, wherein the assay comprises genome sequencing.

6. The method of claim 1, wherein the functional property comprises mass or mass accumulation rate (MAR).

7. The method of claim 6, wherein the mass is measured with a precision of at least about 0.01% of a cell mass.

8. The method of claim 6, wherein the MAR is measured with a precision of at least about 0.1% per hour.

9. The method of claim 3, wherein the measurement instrument comprises a suspended microchannel resonator (SMR).

10. The method of claim 3, wherein a duration of measuring the MAR is from about 20 minutes to about 2 hours.

11. The method of claim 1, wherein the functional property is measured from the live cells within less than about 36 hours of the tissue sample being obtained by biopsy.

12. The method of claim 1, wherein the functional property is measured from the live cells within less than about 3 hours after the disaggregating step.

13. The method of claim 1, wherein tissue sample comprises a fine needle aspirate.

14. The method of claim 1, wherein the tissue sample is obtained from a pleural effusion in a subject.

15. The method of claim 1, wherein the tissue sample is obtained from ascetic fluid in a subject.

16. The method of claim 1, wherein the living cells are cancer cells.

17. The method of claim 16, wherein the tissue sample is obtained from a solid tumor and the tumor is from one selected from the group consisting of a bone, bladder, brain, breast, colon, esophagus, gastrointestinal tract, urinary tract, kidney, liver, lung, nervous system, ovary, pancreas, prostate, retina, skin, stomach, testicles, and uterus of a subject.

18. The method of claim 16, wherein disaggregating the cancer cells comprises physical or mechanical disaggregation.

19. The method of claim 16, wherein disaggregating the cancer cells comprises chemical disaggregation.

20. The method of claim 16, wherein disaggregating the cancer cells comprises proteolytic disaggregation.
